(19) United States
(12) Patent Application Publication
(10) Pub. No.: US 2022/0184413 A1
(43) Pub. Date: Jun. 16, 2022

(54) GROW LIGHT AND OCULAR LIGHT THERAPY ARRANGEMENTS AND METHODS FOR GROWING PLANTS AND FOR TREATING NEUROLOGICAL DISORDERS

(71) Applicant: Norb Lighting, LLC., Nunica, MI (US)

(72) Inventor: David S. MacKenzie, Nunica, MI (US)

(73) Assignee: Norb Lighting, LLC., Nunica, MI (US)

(21) Appl. No.: 17/652,578

(22) Filed: Feb. 25, 2022

Related U.S. Application Data
(63) Continuation-in-part of application No. 17/303,318, filed on May 26, 2021.
(60) Provisional application No. 63/260,169, filed on Aug. 11, 2021.
(60) Provisional application No. 63/030,595, filed on May 27, 2020.

(51) Int. Cl. A61N 5/06; F21V 23/06; F21V 5/00; A01G 7/04

(52) U.S. Cl. CPC A61N 5/0622; F21V 23/06; F21Y 2113/10; A01G 7/045; F21V 5/00

(57) ABSTRACT

A lighting arrangement for treating a user having a neurological disorder and/or for growing plants includes a light emitting device that emits a first light within a first wavelength range of 620 nm to 750 nm at a first intensity, a second light within a second wavelength range of 500 nm to 565 nm, and a second intensity between 30% and 50% of the first intensity, a third light within a third wavelength range of 565 nm to 590 nm and a third intensity between 25% and 40% of the first intensity, and a fourth light within a fourth wavelength range of 435 nm to 500 nm and a fourth intensity equal to or less than 30% of the first intensity.

### CROSS-REFERENCE TO RELATED APPLICATIONS

This application claims the benefit of U.S. Provisional Patent Application No. 63/260,169, entitled “GROW LIGHT AND OCULAR LIGHT THERAPY ARRANGEMENTS AND METHODS FOR GROWING PLANTS AND FOR TREATING NEUROLOGICAL DISORDERS,” filed on Aug. 11, 2021, and the present application is a continuation-in-part of U.S. patent application Ser. No. 17/303,318, entitled “OCULAR LIGHT THERAPY ARRANGEMENT AND METHOD FOR TREATING NEUROLOGICAL DISORDERS,” filed May 26, 2021, which claims the benefit of U.S. Provisional Patent Application No. 63/030,595, entitled “OCULAR LIGHT THERAPY ARRANGEMENT AND METHOD FOR TREATING NEUROLOGICAL DISORDERS,” filed on May 27, 2020, the entire disclosures of which are incorporated herein by reference.

### BACKGROUND

The aspects as disclosed and described herein relate to a grow light arrangement and an ocular light therapy arrangement and methods for utilizing the same, and in particular a lighting arrangement that may be utilized as a grow light arrangement for growing plants indoors and in horticultural settings and/or an ocular light therapy arrangement for treating a user having a neurological disorder, such as migraines, fibromyalgia, neuropathy, chronic headaches, and the like, and methods relating to the same.

### BRIEF SUMMARY

One aspect of the embodiments shown and described herein includes an ocular light therapy arrangement for treating a user having a neurologic disorder, the ocular light therapy arrangement including a light emitting device that emits a first light within a first wavelength range of from about 620 nm to about 750 nm at a first intensity, a second light within a second wavelength range of from about 500 nm to about 565 nm where a second intensity of the second light is within a range of between about 30% and about 50% of the first intensity of the first light, a third light within a third wavelength range from about 565 nm to about 590 nm where a third intensity of the third light is within a range of between about 30% and about 40% of the first intensity of the first light, and a fourth light within a fourth wavelength range of from about 435 nm to about 500 nm where a fourth intensity of the fourth light is less than 30% of the first intensity of the first light.

Another aspect of the embodiments shown and described herein may further or alternatively include a light arrangement that includes a light emitting device that emits a first light within a first wavelength range of from about 680 nm to about 750 nm at a first intensity, a second light within a second wavelength range of from about 510 nm to about 550 nm where a second intensity of the second light is within a range of between about 35% and about 45% of the first intensity of the first light, a third light within a third wavelength range from about 565 nm to about 590 nm where a third intensity of the third light is within a range of between about 25% and about 40% of the first intensity of the first light, and a fourth light within a fourth wavelength range of from about 435 nm to about 500 nm where a fourth intensity of the fourth light is less than 5% of the first intensity of the first light.

Yet another aspect of the embodiments shown and described herein may further or alternatively include a method for treating a neurological disorder of a user with an ocular light therapy arrangement, the method including providing a light emitting device, actuating the light emitting device such that the light emitting device emits a first light within a first wavelength range of from about 620 nm to about 750 nm at a first intensity, a second light within a second wavelength range of from about 500 nm to about 565 nm where a second intensity of the second light is within a range of between about 30% and about 50% of the first intensity of the first light, a third light within a third wavelength range from about 565 nm to about 590 nm where a third intensity of the third light is within a range of between about 25% and about 40% of the first intensity of the first light, and a fourth light within a fourth wavelength range of from about 435 nm to about 500 nm where a fourth intensity of the fourth light is less than 30% of the first intensity of the first light, and positioning the light emitting device such that the lights emitted from the light emitting device illuminates a space occupied by the user, wherein the light is effective to reduce neurologic disorder symptoms by reducing stimulation of the user's retina and brain cortex.

Still yet another aspect of the embodiments shown and described herein may further or alternatively include a method for growing plants that includes providing a light emitting device, actuating the light emitting device such that the light emitting device emits a first light within a first wavelength range of from about 620 nm to about 750 nm at a first intensity, a second light within a second wavelength range of from about 500 nm to about 565 nm where a second intensity of the second light is within a range of between about 30% and about 50% of the first intensity of the first light, a third light within a third wavelength range from about 565 nm to about 590 nm where a third intensity of the third light is within a range of between about 25% and about 40% of the first intensity of the first light, and a fourth light within a fourth wavelength range of from about 435 nm to about 500 nm where a fourth intensity of the fourth light is less than 30% of the first intensity of the first light, providing a plant, and positioning the light emitting device such that the lights emitted from the light emitting device illuminates a space occupied by the plant, wherein the light is effective to develop plant growth.

The present inventive lighting arrangement provides an ocular light therapy arrangement and method and/or a plant grow light arrangement and method. The ocular light therapy arrangement and method is effective for treating users having a neurological disorder, and in particular to reducing the neurological disorder symptoms typically associated with neurological disorders such as migraines, fibromyalgia, neuropathy, chronic headaches, and the like, and provides a relatively low cost treatment for such disorders eliminating or reducing the necessity for medications and, in turn, side effects typically associated with such drugs and medications, eliminating or reducing the necessity for invasive procedures and the side effects and serious risk factors associated therewith, and is particularly well adapted for the proposed use. As a result, the present inventive arrangement and related method may allow the user to resume or conduct normal indoor life/work functions and activities without having to seek refuge and/or recovery in a separate dark or dimly lit room. The plant grow light arrangement and method is effective for nourishing plants and promoting plant growth of plants particularly within indoor and greenhouse settings, and particularly for optimizing development of tropical plants.

These and other features, advantages, and objects of the embodiments as disclosed herein will be further understood and appreciated by those skilled in the art by reference to the following specification, claims, and appended drawings.

### BRIEF DESCRIPTION OF THE DRAWINGS

FIG. 1 is an exploded perspective view of a lighting arrangement;

FIG. 2 is a light spectrum graph of an example of light output of the lighting arrangement;

FIG. 3 is a schematic flow chart of a method for treating a neurological disorder; and

FIG. 4 is a schematic flow chart of a method for growing plants.

### DETAILED DESCRIPTION

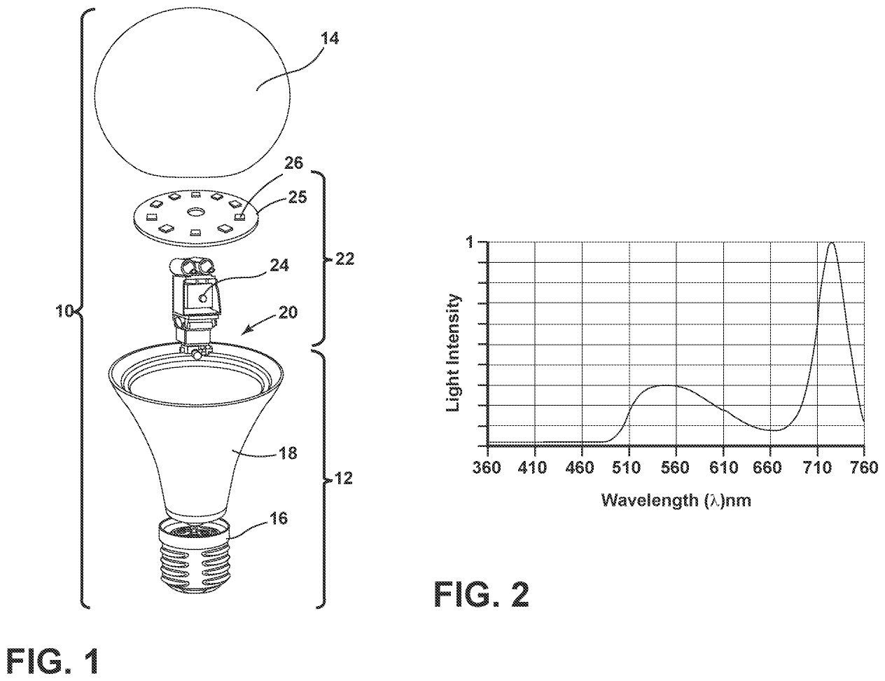

For purposes of description herein, the terms “upper,” “lower,” “right,” “left,” “rear,” “front,” “vertical,” “horizontal,” and derivatives thereof shall relate to the invention as oriented in FIG. 1. However, it is to be understood that the invention may assume various alternative orientations, except where expressly specified to the contrary. It is also to be understood that the specific devices and processes illustrated in the attached drawings, and described in the following specification are simply exemplary embodiments of the inventive concepts defined in the appended claims. Hence, specific dimensions and other characteristics relating to the embodiments disclosed herein are not to be considered as limiting, unless the claims expressly state otherwise.

The reference numeral 10 (FIG. 1) generally represents a lighting arrangement that may be utilized within or as an ocular therapy arrangement and/or a plant grow light as disclosed and described herein. In the illustrated example, the lighting arrangement includes a light emitting device, and in particular, may include a light emitting diode (LED) light bulb. In the illustrated example, the lighting arrangement 10 includes a base assembly 12 and a light diffuser 14, where the base assembly 12 includes a base member 16 and a housing 18, the housing 18 cooperating with the diffuser 14 to form an interior space 20. The lighting arrangement 10 may further include an LED arrangement 22 located within the interior space 20 where the LED arrangement 22 includes an LED driver 24 and an LED array 25 including one or more LEDs 26.

In the illustrated example, the base member 16 includes a standard E26 threaded bulb connector, however, other configurations of the base member 16 may be utilized depending upon the application and connection requirements for the overall lighting arrangement 10.

The LED arrangement 22 is configured such that the lighting arrangement 10 emits a light configured to treat neurological disorders. Specifically, in one embodiment, the light as emitted from the lighting arrangement 10 may be configured to reduce stimulation of the retina and the brain cortex of a user, thereby reducing or eliminating neurological disorder symptoms associated with neurological disorders, such as migraines, fibromyalgia, neuropathy, chronic headaches, and the like. The lighting arrangement 10 may in addition or alternatively be configured to promote plant growth, particularly of tropical plants where tropical plants include those plants that typically grow in tropical climates that are relatively hot and humid with temperatures exceeding 18° C. with zero frost days.

In the illustrated example, the LED array 25 of the lighting arrangement 10 may be configured to produce: a first light in the red light spectrum and preferably within a first wavelength range of between about 620 nm and about 750 nm, more preferably within a range of between 680 nm and 750 nm, and most preferably within a range of between 700 nm and 750 nm, and at a first intensity; a second light in the green light spectrum and preferably within a second wavelength range of between about 500 nm and about 565 nm, more preferably within a range of between 510 nm and 550 nm, and at a second intensity preferably within a range of between about 30% and about 50% of the first intensity, more preferably within a range of between 35% and 45% of the first intensity, and most preferably within a range of between 30% and 40% of the first intensity; a third light in the yellow light spectrum and preferably within a third wavelength range of about between 565 nm and about 590 nm, and more preferably within a wavelength range of between about 570 nm and about 590 nm, and at a third intensity preferably within a range of between about 25% and about 40% of the first intensity, and more preferably within a range of between 25% and 30% of the first intensity; and a fourth light in the blue light spectrum and preferably within a fourth wavelength range of between about 435 nm and about 500 nm, and at a fourth intensity preferably of equal to or less than 30% of the first intensity, more preferably of equal to or less than 5% of the first intensity and most preferably of 3% or less of the first intensity. The light spectrum as described above is illustrated in FIG. 2 showing light wavelength vs. light intensity. It is noted that the combination of the two wavelength ranges, namely, the wavelength ranges within the range of between about 500 nm to about 565 nm and between about 700 nm and 1 mm may simulate the user's perception of natural light filtered through a canopy of foliage.

Further, the lighting arrangement 10 may be configured to have an output of between about 1 lumen and about 1000 lumen, wherein more preferably the output of the lighting arrangement 10 is variable between about 300 lumen and about 600 lumen such that a user can vary the overall intensity of the light as emitted from the lighting arrangement 10. In one example, the light emitting device is configured to have an output of greater than or equal to about 600 lumen.

Still further, the lighting arrangement 10 may further be configured such that the light as emitted has a flicker rate of preferably less than or equal to about 2 Hz, more preferably of less than or equal to about 1 Hz, and most preferably of about zero Hz. The light bulb 10 may also be configured such that the light as emitted has a flicker rate of greater than or equal to about 85,000 Hz in addition to or as an alternative to the light as emitted at the lower flicker rate as described above. The lighting arrangement 10 may further be configured such that the flicker index of the light as emitted from the light bulb 10 is preferably less than or equal to about 2.0, more preferably is less than or equal to about 1.0, and most preferably is less than about 0.02, where the flicker index is a measure of the cyclic variation in output of the light source, taking into account the waveform of the light output, and is specifically the ratio of the area under the light output curve that is above the average light output level to the total area under the light output curve for a single cycle. The lighting arrangement 10 may still further be configured such that the light as emitted from the light bulb 10 preferably has less than or equal to about 6% flicker, more preferably of less than or equal to about 5% flicker, and most preferably of less than or equal to about 2.5%, where the flicker percentage is the measure of the maximum light versus the minimum light in the cycle, and accounts for the minimum and maximum light outputs without differentiating between waveforms.

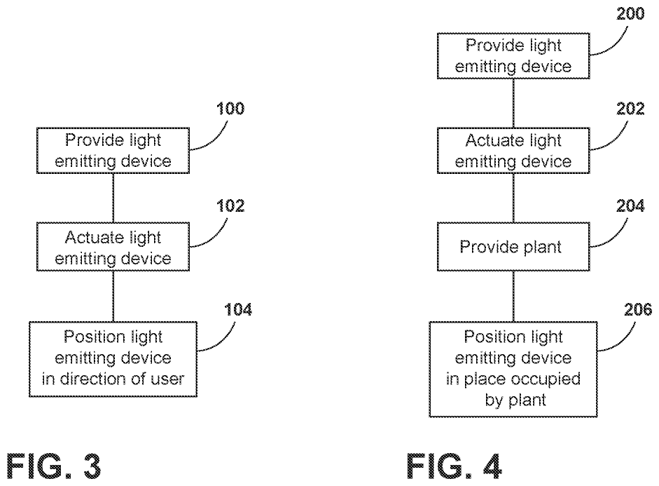

As best illustrated in FIG. 3, a method for utilizing the lighting arrangement 10 as an ocular light therapy arrangement may include providing 100 the light emitting device, namely, the lighting arrangement 10, actuating 102 the lighting arrangement 10 such that the light(s) emitted from the lighting arrangement 10 has the characteristics and is within the parameters as described above, and positioning 104 the lighting arrangement 10 such that the light emitted from the lighting arrangement 10 illuminates a space occupied by the user, such that the light is preferably received by the eyes of a user, thereby reducing stimulation of the user's retina and brain cortex and reducing and/or soothing or preventing the triggering of neurological disorder symptoms typically associated with the neurological disorders such as pain, and as discussed above.

As best illustrated in FIG. 4, a method for utilizing the lighting arrangement 10 as a plant grow light arrangement may include providing 200 the light emitting device, namely, the lighting arrangement 10, actuating 202 the lighting arrangement 10 such that the light(s) emitted from the lighting arrangement 10 has the characteristics and is within the parameters as discussed above providing 204 a plant, and positioning 206 the light arrangement 10 such that the light emitted from the lighting arrangement 10 illuminates a space occupied by a plant, where the light(s) promote plant growth.

The present inventive lighting arrangement provides an ocular light therapy arrangement and method and/or a plant grow light arrangement and method, where the ocular light therapy arrangement is effective for treating users having a neurological disorder, and in particular to reducing the neurological disorder symptoms such as headaches and pain, nausea, vomiting and extreme sensitivity to light and sound, typically associated with neurological disorders such as migraines, fibromyalgia, neuropathy, chronic headaches, and the like, and provides a relatively low cost treatment for such disorders eliminating or reducing the necessity for medications and, in turn, side effects typically associated with such drugs and medications, eliminating or reducing the necessity for invasive procedures and the side effects and serious risk factors associated therewith, and is particularly well adapted for the proposed use, while the plant grow light arrangement and method is effective for nourishing plants and promoting plant growth of plants, particularly in indoor and greenhouse settings, and particularly for optimizing development of tropical plants. As a result, the present inventive arrangement and related method may allow the user to resume or conduct normal indoor life/work functions and activities without having to seek refuge and/or recovery in a separate dark or dimly lit room, and/or may be utilized within the agricultural industry to provide a relatively low cost plant grow light which optimizes the growth of tropical plants.

In the foregoing description, it will be readily appreciated by those skilled in the art that alternative embodiments of the various components and elements of the disclosed embodiments and modifications to the invention may be made without departing from the concepts disclosed herein. Such modifications are to be considered as included in the following claims, unless these claims by their language expressly state otherwise.

The invention claimed is:

1. An ocular light therapy arrangement for treating a user having a neurologic disorder, comprising:
a light emitting device that emits a first light within a first wavelength range of from about 620 nm to about 750 nm at a first intensity, a second light within a second wavelength range of from about 500 nm to about 565 nm where a second intensity of the second light is within a range of between about 30% and about 50% of the first intensity of the first light, a third light within a third wavelength range from about 565 nm to about 590 nm where a third intensity of the third light is within a range of between about 25% and about 40% of the first intensity of the first light, and a fourth light within a fourth wavelength range of from about 435 nm to about 500 nm where a fourth intensity of the fourth light is equal to or less than 30% of the first intensity of the first light.

2. The ocular light therapy arrangement of claim 1, wherein the first wavelength range is between 680 nm and 750 nm.

3. The ocular light therapy arrangement of claim 2, wherein the first wavelength range is between 700 nm and 750 nm.

4. The ocular light therapy arrangement of claim 1, wherein the second wavelength range is between 510 nm and 550 nm.

5. The ocular light therapy arrangement of claim 1, wherein the third wavelength range is between 570 nm and 590 nm.

6. The ocular light therapy arrangement of claim 1, wherein the fourth wavelength range is between 435 nm and 500 nm.

7. The ocular light therapy arrangement of claim 1, wherein the second intensity of the second light is between about 35% and about 45% of the intensity of the first light.

8. The ocular light therapy arrangement of claim 7, wherein the second intensity of the second light is between about 30% and about 40% of the intensity of the first light.

9. The ocular light therapy arrangement of claim 1, wherein the third intensity of the third light is between about 25% and about 30% of the intensity of the first light.

10. The ocular light therapy arrangement of claim 1, wherein the intensity of the fourth light is equal to or less than 5% of the first intensity.

11. The ocular light therapy arrangement of claim 10, wherein the fourth intensity of the fourth light is equal to or less than 3% of the first intensity.

12. The ocular light therapy arrangement of claim 1, wherein the light emitting device comprises at least one light emitting diode.

13. The ocular light therapy arrangement of claim 1, further comprising:
a base member configured to be releasably received within a light bulb socket;
a housing member coupled to the base member; and
a diffuser coupled to the base member and cooperating with the base member to define an interior space, wherein the light emitting device is located within the interior space.

14. The ocular light therapy arrangement of claim 1, wherein the neurologic disorder symptoms include neurologic disorder symptoms of a migraine.

15. A lighting arrangement, comprising:
a light emitting device that emits a first light within a first wavelength range of from about 680 nm to about 750 nm at a first intensity, a second light within a second wavelength range of from about 510 nm to about 550 nm where a second intensity of the second light is within a range of between about 35% and about 45% of the first intensity of the first light, a third light within a third wavelength range from about 565 nm to about 590 nm where a third intensity of the third light is within a range of between about 25% and about 40% of the first intensity of the first light, and a fourth light within a fourth wavelength range of from about 435 nm to about 500 nm where a fourth intensity of the fourth light is less than 5% of the first intensity of the first light.

16. The lighting arrangement of claim 15, wherein the first wavelength range is between 700 nm and 750 nm.

17. The lighting arrangement of claim 16 wherein the second wavelength range is between 510 nm and 550 nm.

18. The lighting arrangement of claim 15, wherein the second intensity of the second light is between about 30% and about 40% of the intensity of the first light.

19. The lighting arrangement of claim 15, wherein the third intensity of the third light is between about 25% and about 30% of the intensity of the first light.

20. The lighting arrangement of claim 15, wherein the fourth intensity of the fourth light is equal to or less than 3% of the first intensity.

21. The lighting arrangement of claim 20, wherein the fourth intensity of the fourth light is about zero.

22. The lighting arrangement of claim 15, wherein the first wavelength range is between 700 nm and 750 nm, the second intensity of the second light is between 30% and 40% of the first intensity of the first light, the third wavelength is between 570 nm and 580 nm and the third intensity of the third light between 25% and 30% of the first intensity of the first light, and wherein the fourth intensity of the fourth light is less than or equal to about 3% of the first intensity.

23. The lighting arrangement of claim 15, wherein the light emitting device comprises at least one light emitting diode.

25. The lighting arrangement of claim 15, further comprising:
a base member configured to be releasably received within a light bulb socket;
a housing member coupled to the base member; and
a diffuser coupled to the base member and cooperating with the base member to define an interior space, wherein the light emitting device is located within the interior space.

26. An ocular light therapy arrangement for treating a user having a neurological disorder, comprising the lighting arrangement of claim 15, wherein the neurologic disorder symptoms include neurologic disorder symptoms of a migraine.

27. A plant grow light arrangement for growing plants, comprising the lighting arrangement of claim 15, wherein the grow light arrangement is configured to promote plant growth.

28. A method for treating a neurological disorder of a user with an ocular light therapy arrangement, comprising:
providing the light emitting device of claim 1;
actuating the light emitting device such that the light emitting device emits the first light within the first wavelength range at the first intensity, the second light within the second wavelength range where the second intensity of the second light is within the range of between about 30% and about 50% of the first intensity of the first light, the third light within the third wavelength range where the third intensity of the third light is within the range of between about 25% and about 40% of the first intensity of the first light, and the fourth light within the fourth wavelength range where the fourth intensity of the fourth light is less than 30% of the first intensity of the first light; and
positioning the light emitting device such that the lights emitted from the light emitting device illuminate a space occupied by the user, wherein the light is effective to reduce neurologic disorder symptoms by reducing stimulation of the user's retina and brain cortex.

29. A method for growing plants, comprising:
providing a light emitting device of claim 15;
actuating the light emitting device such that the light emitting device emits the first light within the first wavelength range at the first intensity, the second light within the second wavelength range where the second intensity of the second light is within the range of between about 30% and about 50% of the first intensity of the first light, the third light within the third wavelength range where the third intensity of the third light is within the range of between about 25% and about 40% of the first intensity of the first light, and the fourth light within the fourth wavelength range where the fourth intensity of the fourth light is less than 30% of the first intensity of the first light;
providing a plant; and
positioning the light emitting device such that the lights emitted from the light emitting device illuminate a space occupied by the plant, wherein the lights are configured to promote plant growth.
